(19) United States
(12) Patent Application Publication
(10) Pub. No.: US 2025/0099769 A1
(43) Pub. Date: Mar. 27, 2025

(54) Systems And Methods For Stellate Ganglion Stimulation And Ablation

(71) Applicant: Mayo Foundation for Medical Education and Research, Rochester, MN (US)

(72) Inventors: Yong-Mei CHA, Rochester, MN (US); Samuel J. ASIRVATHAM, Rochester, MA (US)

(73) Assignee: Mayo Foundation for Medical Education and Research, Rochester, MN (US)

(21) Appl. No.: 18/666,487

(22) Filed: May 16, 2024

Related U.S. Application Data
(63) Continuation of application No. 17/241,523, filed on Apr. 27, 2021, now abandoned.
Application No. 17/241,523 is a division of application No. 16/804,878, filed on Feb. 28, 2020, now abandoned.
(60) Provisional application No. 62/815,584, filed on Mar. 8, 2019.

(51) Int. Cl. A61N 1/36; A61N 1/05; A61N 1/32; A61N 1/365

(52) U.S. Cl. CPC A61N 1/36114; A61N 1/0551; A61N 1/0573; A61N 1/327; A61N 1/36034; A61N 1/36564

(57) ABSTRACT

This document relates to methods and materials for providing stimulation or ablation to the stellate ganglion. For example, this document relates to methods and devices for providing stimulation or ablation to the stellate ganglion to modify blood pressure.

### CROSS-REFERENCE TO RELATED APPLICATIONS

This application is a continuation of U.S. patent application Ser. No. 17/241,523 filed Apr. 27, 2021, which is a divisional of U.S. patent application Ser. No. 16/804,878 filed Feb. 28, 2020, which claims the benefit of U.S. Provisional Application Ser. No. 62/815,584, filed Mar. 8, 2019. The disclosure of the prior applications is considered part of (and is incorporated by reference in) the disclosure of this application.

### BACKGROUND

### 1. Technical Field

This document relates to methods and materials for providing stimulation or ablation to the stellate ganglion. For example, this document relates to methods and devices for providing stimulation or ablation to the stellate ganglion to modify blood pressure.

### 2. Background Information

Hypertension, commonly known as high blood pressure, is a long-term condition in which the blood pressure is persistently elevated and can affect 16-37% of the population globally. Long-term high blood pressure can be a major risk factor for coronary artery disease, stroke, heart failure, peripheral vascular disease, vision, and chronic kidney disease, to name a few. Lifestyle changes and medications can lower blood pressure and decrease the risk of health complications. Lifestyle changes can include weight loss, decreased salt intake, physical exercise, and a healthy diet. If lifestyle changes are not sufficient, then blood pressure medications can be used.

Syncope, commonly known as fainting, is a loss of consciousness and muscle strength characterized by a fast onset, short duration, and spontaneous recovery and can account for about three percent of visits to emergency departments, affect about three to six of every thousand people each year. Fainting can be caused by a decrease in blood flow to the brain, usually from low blood pressure. Treatment can include returning blood to the brain by positioning the person on the ground, with legs slightly elevated or leaning forward and the head between the knees. For individuals who have problems with chronic fainting spells, therapy can focus on recognizing the triggers and learning techniques to keep from fainting. At the appearance of warning signs, such as lightheadedness, nausea, or cold and clammy skin, counter-pressure maneuvers that can include gripping fingers into a fist, tensing the arms, and crossing the legs or squeezing the thighs together can be used to ward off a fainting spell. Further, vasovagal syncope (VVS) is the leading cause of syncope, especially in the absence of cardiac disease. The mechanism of syncope is characterized as either a cardioinhibitory response, a vasodepressor response or, most commonly, a mixture of both. In some instances, there may be concomitant autonomic dysfunction.

The autonomic nervous system controls most of the involuntary reflexive activities of the human body. The system is constantly working to regulate the glands and many of the muscles of the body through the release or uptake of the neurotransmitters acetylcholine and norepinephrine. Autonomic dysregulation involves malfunctioning of the autonomic nervous system, the portion of the nervous system that conveys impulses between the blood vessels, heart, brain, and all the organs in the chest, abdomen, and pelvis.

### SUMMARY

This document describes methods and materials for providing stimulation or ablation to the stellate ganglion. For example, this document describes methods and devices for providing stimulation or ablation to the stellate ganglion to modify blood pressure.

In one aspect, this disclosure is directed to a method of modulating hemodynamic parameters of a patient. The method includes positioning a device with a first electrode proximal one of a stellate ganglion or a subclavius ansa and delivering stimulation via the first electrode to one of the stellate ganglion or the subclavius ansa. In some cases, delivering stimulation via the first electrode can include delivering stimulation with a first set of stimulation parameters to increase a blood pressure of the patient. In some cases, delivering stimulation via the first electrode can include delivering stimulation with a second set of stimulation parameters to decrease a blood pressure of the patient. In some cases, the method can include securing the device proximal to one of the stellate ganglion or the subclavius ansa. In some cases, securing the device can include at least one of screwing a portion of the device into one of the stellate ganglion or the subclavius ansa, screwing a portion of the device into tissue proximal one of the stellate ganglion or the subclavius ansa, securing the device proximal one of the stellate ganglion or the subclavius ansa via a barb, securing the device proximal one of the stellate ganglion or the subclavius ansa via a hook, or clamping a portion of the device around one of the stellate ganglion or the subclavius ansa. In some cases, the method can include coupling a proximal portion of the device to a stimulation generator.

In some cases, the device can include a second electrode distal the first electrode, and the method can include positioning the second electrode proximal a portion of a heart of the patient. In some cases, the method can include delivering stimulation via the second electrode. In some cases, the method can include sensing a change in blood pressure of the patient via the first electrode. In some cases, delivering stimulation via the first electrode to one of the stellate ganglion or the subclavius ansa can include delivering stimulation via the first electrode to one of the stellate ganglion or the subclavius ansa in response to the change in blood pressure of the patient. In some cases, the method can include sensing a blood pressure of the patient via the second electrode. In some cases, delivering stimulation via the first electrode to one of the stellate ganglion or the subclavius ansa can include delivering stimulation via the first electrode to one of the stellate ganglion or the subclavius ansa in response to the change in blood pressure of the patient.

In some cases, the method can include sensing a blood pressure. In some cases, sensing the blood pressure can include sensing the blood pressure via the first electrode. In some cases, delivering stimulation via the first electrode to one of the stellate ganglion or the subclavius ansa can include delivering stimulation via the first electrode to one of the stellate ganglion or the subclavius ansa in response to a change in the blood pressure of the patient. In some cases, sensing the blood pressure can include sensing the blood pressure via one of a pressure sensor or plethysmograph. In some cases, delivering stimulation via the first electrode to one of the stellate ganglion or the subclavius ansa can include delivering a stimulatory sequence. In some cases, the method can include recording a response to the stimulatory sequence. In some cases, the method can include determining an increase or a decrease in activity in response due to the stimulatory sequence. In some cases, the method can include determining if the device was positioned in a correct direction based on the increase or the decrease in activity.

Particular embodiments of the subject matter described in this document can be implemented to realize one or more of the following advantages. First, stimulation of the stellate ganglion and/or ansa subclavius can significantly change (e.g., increase) hemodynamic parameters, such as systolic blood pressure, diastolic blood pressure, and heart rate. Second, stimulation of the stellate ganglion and/or ansa subclavius can produce significant changes in hemodynamic parameters despite background high output vagal stimulation. This can be especially beneficial during times of excess vagal tone, such as during vasovagal syncope. Third, the length of the ansa subclavius provides a greater target size, and multiple anatomic vantage points to which a lead can be secured. Fourth, a loop or remote suture electrode can be used to place over the stellate ganglion and/or ansa subclavius, or more than one electrode can be targeted along the stellate ganglion and/or ansa subclavius for redundancy and/or diagnostics. Fifth, there are two stellate ganglion such that treatment can be provided to one, or both of the stellate ganglion. Sixth, electroporation and/or ablation can be used to decrease blood pressure, and/or stop an arrhythmia.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the invention are set forth in the accompanying drawings and the description below. Other features, objects, and advantages of the invention will be apparent from the description, drawings, and from the claims.

### DESCRIPTION OF DRAWINGS

FIG. 1 shows the anatomy of and around the stellate ganglion in accordance with some embodiments provided herein.

FIG. 2 shows percutaneous placement of a wire on the stellate ganglion using a posterior approach in accordance with some embodiments provided herein.

FIG. 3 shows percutaneous placement of a wire on the stellate ganglion using an anterior approach in accordance with some embodiments provided herein.

FIG. 4 shows placement of a mesh stent in a subclavian vein near the stellate ganglion in accordance with some embodiments provided herein.

FIG. 5 shows a wire placed around the stellate ganglion in accordance with some embodiments provided herein.

Like reference numbers represent corresponding parts throughout.

### DETAILED DESCRIPTION

This document describes methods and materials for providing stimulation or ablation to the stellate ganglion. For example, this document describes methods and devices for providing stimulation or ablation to the stellate ganglion to modify blood pressure.

The autonomic nervous system controls most of the involuntary reflexive activities of the human body. The system is constantly working to regulate the glands and many of the muscles of the body through the release or uptake of the neurotransmitters acetylcholine and norepinephrine. Autonomic dysregulation involves malfunctioning of the autonomic nervous system, the portion of the nervous system that conveys impulses between the blood vessels, heart, brain, and all the organs in the chest, abdomen, and pelvis. Accordingly, neural stimulation that negates or overcomes the effects of vagal tone may be used as a treatment strategy for vasovagal syncope (VVS or hypertension).

Particular embodiments of the subject matter described in this document can be implemented to realize one or more of the following advantages. First, stimulation of the stellate ganglion can significantly change (e.g., increase) hemodynamic parameters, such as systolic blood pressure, diastolic blood pressure, and heart rate. Second, stimulation of the stellate ganglion can produce significant changes in hemodynamic parameters despite background high output vagal stimulation. This can be especially beneficial during times of excess vagal tone, such as during vasovagal syncope. Third, the length of the ansa subclavius provides a greater target size, and multiple anatomic vantage points to which a lead can be secured. Fourth, a loop or remote suture electrode can be used to place over the ansa subclavius, or more than one electrode can be targeted along the ansa subclavius for redundancy and/or diagnostics.

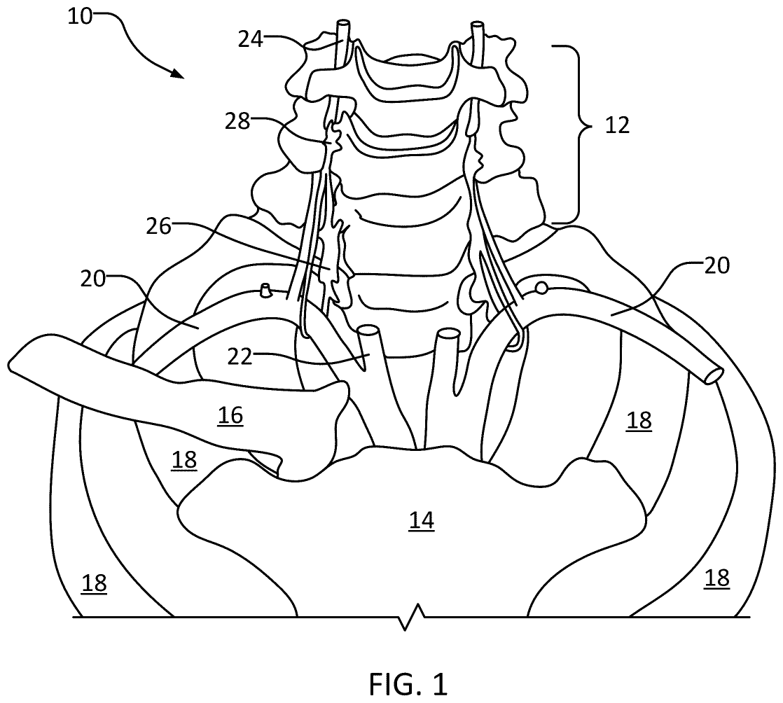

Referring to FIG. 1, a body 10 can include bones, blood vessels, and nerves, among other anatomy. The bones of body 10 can include a vertebral column 12 extending along a back of the body, a sternum 14 located in the center of the chest, connecting ribs 18 via cartilage. Also shown is a clavicle 16, extending from the sternum 14.

The blood vessels of body 10 can include subclavian arteries 20, common carotid arteries 22, and vertebral arteries 24. Subclavian arteries 20 are paired major arteries of the upper thorax, below clavicle 16, and receive blood from the aortic arch. The left subclavian artery supplies blood to the left arm and the right subclavian artery supplies blood to the right arm. Common carotid arteries 22 are arteries that supply the head and neck with oxygenated blood; they divide in the neck to form the external and internal carotid arteries. Vertebral arteries 24 are major arteries of the neck. Typically, the vertebral arteries originate from the subclavian arteries 20. Each vessel courses superiorly along each side of the neck, merging within the skull to form the single, midline basilar artery. Vertebral arteries 24 provide supply blood to the upper spinal cord, brainstem, cerebellum, and posterior part of brain.

The nerves of body 10 can include stellate ganglion 26 and middle cervical ganglion 28.

Stellate ganglion 26 (or cervicothoracic ganglion) are sympathetic ganglions formed by the fusion of the inferior cervical ganglion and the first thoracic ganglion. Stellate ganglion 26 are relatively big (10−12×8−20 mm) compared to much smaller thoracic, lumbar, and sacral ganglia and are polygonal in shape (Latin stellatum meaning star-shaped). Stellate ganglion 26 are located at the level of C7, anterior to the transverse process of C7 and the neck of the first rib 18, superior to the cervical pleura and just below the subclavian artery 20. Stellate ganglion 26 are superiorly covered by the prevertebral lamina of the cervical fascia and anteriorly in relation with common carotid artery 22, subclavian artery 20 and the beginning of vertebral artery 24 which sometimes leaves a groove at the apex of stellate ganglion 26.

Middle cervical ganglion 28 is the smallest of the three cervical ganglia, and is occasionally absent. Middle cervical ganglion 28 is placed opposite the sixth cervical vertebra, usually in front of, or close to, the inferior thyroid artery.

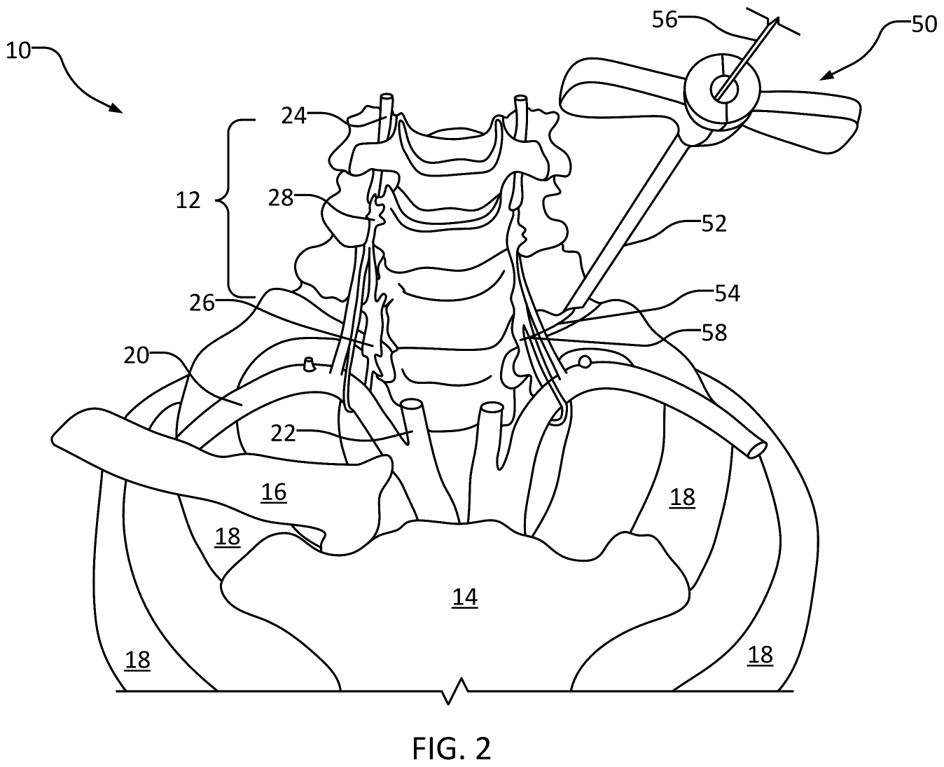

Referring to FIG. 2, a device 50 can include a sheath 52 and a wire 54. Wire 54 can include a proximal portion 56 and a distal portion 58. Device 50 can be used to percutaneously place distal portion 58 of the wire 54 on and/or near stellate ganglion 26 using a posterior approach. In some embodiments, ultrasound imaging of the neck can be used to located stellate ganglion 26.

Wire 54 can pass through sheath 52, such that sheath 52 is an oversheath. In some embodiments, sheath 52 can be deflectable. In some embodiments, a deflectable catheter can be placed into sheath 52, and wire 54 can pass through the deflectable catheter. Sheath 52 and wire 54 can be advance through body 10 until stellate ganglion 26 is reached. In some embodiments, device 50 can enter body 10 at a posterior lateral side of a neck of a patient. In some embodiments, once stellate ganglion 26 is reached, stimulation can be provided to stellate ganglion 26 via distal portion 58 of wire 54, to confirm the location of stellate ganglion 26 and check for any safety issues. In some cases, the deflectable catheter can be used to advance wire 54, and once stellate ganglion 26 is reached, sheath 52 can be advanced to secure a position. In some embodiments, deflectable catheter can provide stimulation while being advanced to stellate ganglion 26, sheath 52 can then be advanced to secure a position, and then wire 54 can be advanced to be in contact with stellate ganglion 26.

In some embodiments, sheath 52 can include a mechanism to secure sheath 52 to stellate ganglion 26. For example, the mechanism can be a helix, a tine, a harp, or other means for securing sheath 52 to stellate ganglion 26. In some cases, wire 54 can include a small insulated clip that can be used to anchor and/or secure distal portion 58 of wire 54 to stellate ganglion 26. In some cases, the small insulated clip can aid in preventing stimulation of the intercostal muscle, which can cause fasciculation and an immediate rise in blood pressure. In some cases, the small insulated clip can have an uninsulated interior that is capable of stimulating stellate ganglion 26 and an insulated exterior, such that no stimulation occurs to surrounding muscle. In some cases, the small insulated clip is a portion of distal portion 58 of wire 54, such that the small insulated clip is connected to a subcutaneous stimulation generator. In some cases, proximal portion 56 of wire 54 can be insulated and tunneled to a subcutaneous area. For example, proximal portion 56 of wire 54 can be connected to a subcutaneous stimulation generator.

In some embodiments, wire 54 can deliver high frequency stimulation. For example, stimulation can be delivered at about 5-15 Hz, or about 10 Hz. As another example, stimulation can be delivered with a pulse width of about 1-3 ms, or about 2 ms. When stimulation is provided to stellate ganglion 26, a rise in heart rate and/or blood pressure will be observed.

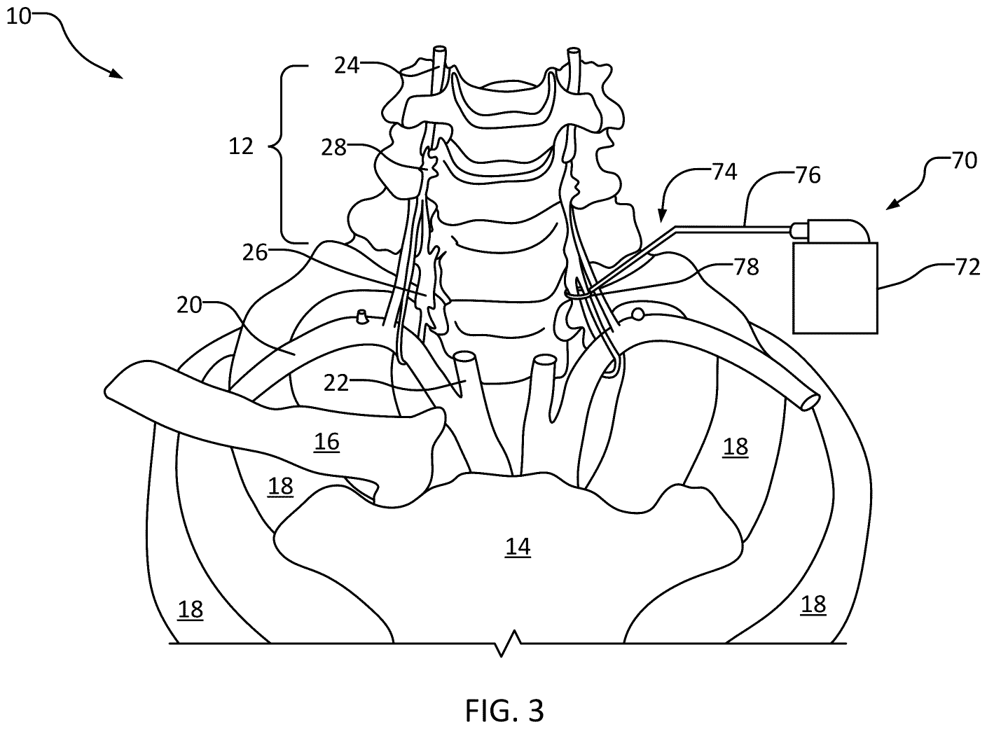

Referring to FIG. 3, a device 70 can include a stimulation generator 72 and a wire 74. A proximal portion 76 of wire 74 can be connected to stimulation generator 72. Stimulation generator 72 can be implanted subcutaneously to cause stimulation of wire 74. Device 70 can be percutaneously placed such that a distal portion 78 of the wire 74 on and/or near stellate ganglion 26 using an anterior approach. In some embodiments, an optical scope (e.g., an ultrasound scope) can be used to located stellate ganglion 26.

During implantation of device 70, a needle can be percutaneously inserted into body 10 until a region including stellate ganglion 26 is reached. A spreading tool (e.g., a dilator) can be advanced over the needle until stellate ganglion 26 is reached. In some embodiments, multiple spreading tools can be used. For example, a first spreading tool can be passed over the needle, then a second, larger spreading tool can be passed over the first spreading tool. In some embodiments, the spreading tool can include one or more electrodes to provide stimulation. In some embodiments, a sheath can be passed over the spreading tool. In some embodiments, the sheath can be visualized using ultrasound. In some embodiments, a light source and scope is used with the sheath to identify structures. In some embodiments, the direct visualization can aid in confirming device 70 is at the correct location (e.g., at or near stellate ganglion 26). Such direct visualization can be advantageous because the ansa subclavia can be difficult to see using ultrasound imaging techniques.

Wire 74 can be advanced through the spreading tool and/or the sheath and secured at or near stellate ganglion 26. Distal portion 78 of wire 74 can be in contact with stellate ganglion 26. In some embodiments, distal portion 78 of wire 74 can loop around stellate ganglion 26. In some embodiments, a small insulated clip can cover distal portion 78 of wire 74 and stellate ganglion 26.

In some embodiments, wire 74 can deliver high frequency stimulation. In some embodiments, the sheath can provide high frequency stimulation. For example, stimulation can be delivered at about 5-15 Hz, or about 10 Hz. As another example, stimulation can be delivered with a pulse width of about 1-3 ms, or about 2 ms. When stimulation is provided to stellate ganglion 26, a rise in heart rate and/or blood pressure will be observed.

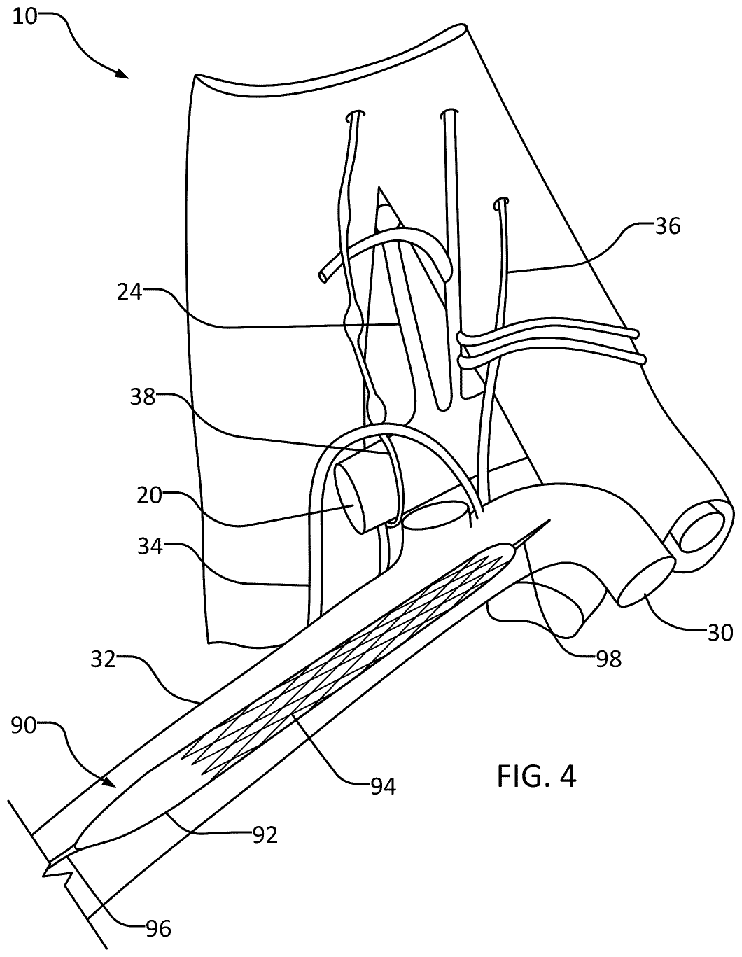

Referring to FIG. 4, body 10 can includes a subclavia ansa 38 (e.g., a subclavian loop), a phrenic nerve 36, a thoracic duct 34, a left brachiocephalic vein 32, and a subclavian vein 30. Subclavia ansa 38 is a nerve cord that is a connection between the middle and inferior cervical ganglion which is commonly fused with the first thoracic ganglion, which is then called the stellate ganglion (as shown in FIGS. 1-3). Subclavia ansa 38 forms a loop around the subclavian artery 20 from anterior to posterior and then lies medially to the internal thoracic artery. Subclavian vein 30 can be located next to subclavian artery 20.

Accordingly, a device 90 can be inserted into subclavian vein 30 and is capable of delivering electrical pulses to stimulate subclavian ansa 38. Device 90 can include a balloon 92, a mesh stent 94, a catheter 96, and a needle 98.

In some embodiments, needle 98 is passed into subclavian vein 30 until a desired location is reached. Catheter 96 can be passed over needle 98 to reach the desired location of subclavian vein 30. In some embodiments, device 90 and the methods of implanting mesh stent 94 can be used in a jugular vein.

Balloon 92 can be mounted on a distal portion of catheter 96. In some cases, catheter 96 can be a deflectable catheter. In some embodiments, balloon 92 can be a circumferential balloon that is in contact with a wall of subclavian vein 30 when inflated. In some cases, a center portion of balloon 92 can be open, such that balloon 92 is open to blood flow.

Mesh stent 94 can be mounted on an exterior of balloon 92. In some cases, mesh stent 94 is expandable, such that expansion of balloon 92 causes expansion of mesh stent 94. Mesh stent 94 can include an electrode. In some cases, mesh stent 94 can include a plurality of electrodes. Optionally, the plurality of electrodes can be positioned longitudinally along mesh stent 94, circumferentially around mesh stent 94, or a combination thereof. For example, mesh stent 94 can include circular rings of electrodes. In some cases, mesh stent 94 can include 5-30 circular rings of electrodes.

The electrode(s) on mesh stent 94 can deliver electrical pulses. During implantation of mesh stent 94, the electrode(s) can deliver electrical pulses until a change in heart rate and/or blood pressure is detected (e.g., via an external sensor on body 10). The change in heart rate and/or blood pressure can indicate mesh stent 94 is at a location of subclavian vein 30 such that subclavian ansa 38 is stimulated from the electrical pulses delivered by the electrode(s) of mesh stent 94.

In some embodiments, the electrodes on mesh stent 94 can be stimulated sequentially (e.g., across a longitudinal axis of mesh stent 94, across circular rings of electrodes) and the heart rate and/or blood pressure of a patient can be monitored. When a desired change in heart rate and/or blood pressure is obtained, a location for mesh stent 94 can be determined. In some cases, mesh stent 94 can maintain the location that provided the desired effects, and only the electrodes that provided the desired change in heart rate and/or blood pressure will continue to provide stimulation. In some cases, mesh stent 94 can be repositioned such that a plurality of electrodes can provide stimulation the results in the desired change in heart rate and/or blood pressure. In some cases, mesh stent 94 can be secured in place at a location that provides the desired change in heart rate and/or blood pressure. In some cases, mesh stent 94 can be secured in place by expanding until mesh stent 94 abuts a wall of subclavian vein 30. In some embodiments, mesh stent 94 can be secured in muscular tissue.

In some embodiments mesh stent 94 can be connected to a stimulation generator. The stimulation generator can be implanted subcutaneously and cause electrical stimulation of electrodes on mesh stent 94. In some cases, catheter 96 is only used for implantation of mesh stent 96, and a wire leads from mesh stent 94 to stimulation generator. In some cases, catheter 96 allows a sheath with balloon 92 to be passed over catheter 96 to allow implantation of mesh stent 94 and removal of balloon 92 after implantation.

In some cases, patients who benefit from implantation of device 90 to provide stimulation to subclavian ansa 38 also benefit from a pacing device (e.g., a pacemaker). In some cases, a lead for the pacing device can include mesh stent 94, such that a distal end of the lead is located in the heart and a proximal portion extends through the subclavian vein 30 and includes mesh stent 94. Optionally, both the pacing device and mesh stent 94 can be connected to a single stimulation generator. In some cases, the pacing device has a first lead connected to the stimulation generator while mesh stent 94 has a second lead connected to the stimulation generator.

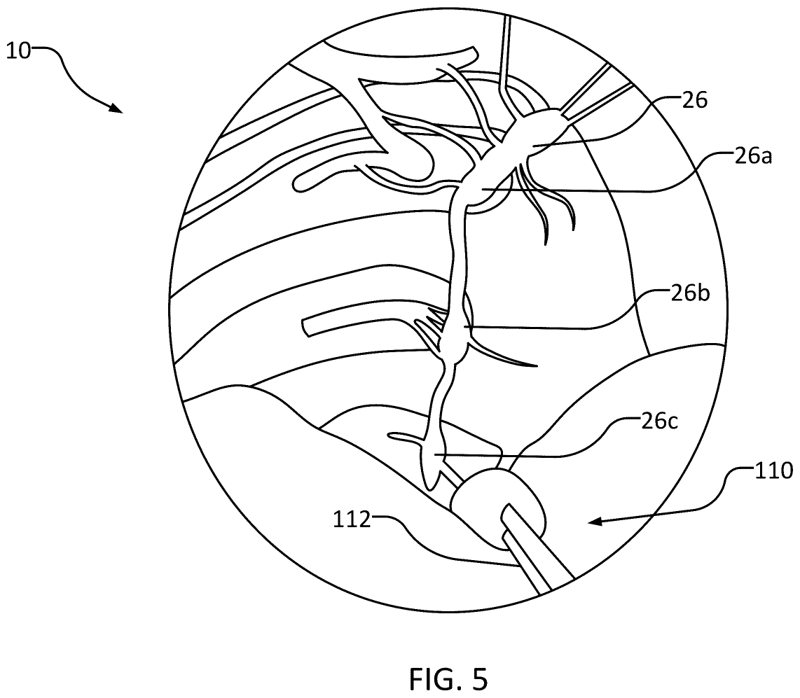

Referring to FIG. 5, a device 110 can be implanted near stellate ganglion 36 using video-assisted thorascopic surgery (VATS). For left cervico-thoracic sympathectomy, a patient is placed in a right lateral position using single lung ventilation. Three 1 cm incisions are made in the sub axillary region for introduction of thoracoscopic instruments. The stellate and thoracic ganglia are located behind the parietal pleura, in the paravertebral position. Stellate ganglion 26, T1 ganglion 26a, T2 ganglion 26b, and/or T3 ganglion 26c can be dissected and completely visualized. Optionally, the pleura can be accessed with two sites, one for a scope, and one for device 110.

In some cases, a lead can be screwed into stellate ganglion 26 and/or tissue surrounding stellate ganglion 26. In some cases, a circumferential soft and flat wire is placed around a portion of stellate ganglion 26. In some cases, the wire is mounted on an insulating band. In some cases, insulating band can prevent electrical current from the wire from leaking to the surrounding musculature.

A wire 112 can be coupled to the lead and/or the circumferential wire. In some cases, wire 112 can be completely insolated and tunneled subcutaneously to a stimulation generator. Optionally, wire 112 can come out through the intercostal space to tunnel to the stimulation generator.

Referring to the figures generally, in some cases, a standard subxiphoid procedure can be used to access the pericardial space and a catheter can be inserted into the pericardium or mediastinum. Then the catheter can be navigated to the stellate ganglion and/or subclavius ansa. In some embodiments, fluoroscopy can be used to navigate the catheter. Once the stellate ganglion and/or subclavius ansa is identified, an electrode can be attached at or near the stellate ganglion and/or subclavius ansa. In some cases, the electrode can be attached via a screw-in member, a needle, a hook, a barb, or a clamp that goes around the stellate ganglion and/or subclavius ansa. A proximal portion of the electrode (e.g., a lead) can be tunneled through the pericardium and to a stimulation generator positioned under the skin of the patient.

Referring to FIGS. 1-5 generally, for safe and consistent deployment of the devices beyond traditional imaging modalities and indeed direct vision because of the uniquely sensitive and crowded topographic environment in which the stellate ganglia lives, the energy delivery tool should be secured to the correct structure. Accordingly, a sensing and effector limb finding tool can be used during implantation of the devices of FIGS. 1-5. A recording algorithm can be used such that neural activity can be sensed, amplified, and recorded, and a template based on direct surgical recordings done previously is used to filter out ambient noise appropriately with widening of the dynamic range and increasing the sampling frequency. As the tool is advanced, candidate signals, when recorded, are tested by delivering a stimulatory sequence. If the recorded signals do respond to the stimulatory sequence by showing an increase or decrease in activity and possibly a change in blood pressure (measured through a plethysmograph or pressure sensor or any of the others detailed below) then noise is excluded and the correct direction of deployment is determined. Such testing can aid in confirming placement when multiple sensors are providing mismatching data. The delivery tool can then be fed in the direction where the now diagnosed and validated correct signal increases in amplitude and near-field nature (slew). The tool either self-navigates or is manually placed at the site where the maximal characteristics of near-field, high amplitude neural signals were identified. At this point, another stimulatory sequence is delivered, sensor crosschecks with any other visualization tools done, and the device is deployed (via the deployment techniques described above).

Optionally, an electrode can be a sensor. In some cases, the devices can include an integrated sensor. In some cases, the sensor can be a pressure sensor or a plethysmogram. The devices can include more than one sensor. In some embodiments, the sensor can monitor neural activity. In some cases, the sensor can be a standalone sensor or a cross-check sensor. Stimulation can be provided until a “baseline” blood pressure is sensed by the sensor.

In some embodiments, the electode(s) of the devices described above can provide stimulation pulses. In some embodiments, the electode(s) of the devices described above can provide inhibitory pulses. In some cases, frequency determines stimulation pulses or inhibitory pulses. Inhibitory pulses can include electroporation. In some cases, electroporation can be reversible. Inhibitor pulses can optionally inhibit neural activity. In some cases, the inhibition of neural activity can be temporary. The pulses can be used to treat high blood pressure and low blood pressure. In some cases, the same electrode can be used for stimulation and electroporation. Optionally, different pulse widths, frequency, and/or output voltage can modify the pulses to be stimulatory or inhibitory.

In some embodiments, the sensor can be mode unique, such as when the device includes a lead to the heart. For example, a sensor on the lead in the heart can detect a change in blood pressure. This sensor can be used to initiate stimulation at the stellate ganglia and/or the ansa subclavius. In some cases, multiple sensors can be used. For example, one sensor can be a primary sensor, and a second sensor can be used as a cross-check. In some cases, the sensor are positioned in different locations (e.g., in the heart and in a blood vessel). In some embodiments, a bifurcated effector arm can be used. The bifurcated effector arm can be used for hypertension, or as a safety mechanism to confirm changes in blood pressure. In some cases, a blood pressure sensor can be located around or adjacent to an artery to determine changes in blood pressure. In devices such as that of FIG. 4, a subclavian artery can be monitored by a sensor to determine changes in blood pressure. Optionally, a vein close to the artery may be able to have a sensor (e.g., part of the mesh).

In some embodiments, a feedback system can be used with the various devices described above. In some cases, the feedback system can be unique to neural structures (e.g., the stellate ganglia and/or the ansa subclavius) and include feedback dose titration.

The delivery systems for the various devices can include three or more bipoles (e.g., a distal bipole, a central bipole, and a proximal bipole). The central bipole can be used for effector therapy such as a neural blockade, ablation, or stimulation. The proximal or upstream pair of electrodes monitors to validate neural signals and forms the feed-forward arm to titrate energy delivery. The distal or downstream electrode pair forms the sensor-check arm to determine whether delivery was sufficient to affect neural function, and then feeds the gathered information to the primary-sensor arm (with upstream electrodes confirming effect or lack thereof). This constant feedback dose titration can allow much lower outputs of stimulation without the need for a safety margin for paced output and can include two important sequelae of practical value. First, lower energy delivery capability can be important in preventing phrenic nerve stimulation, sensory nerve stimulation, pain, and muscle twitching. Second, continuous modulated therapy can be delivered, which can be more effective than one-time effector therapy for blood pressure control and management of autonomic dysfunction.

In some embodiments, the feedback system can include templates for a “normal” sensor reading. In some embodiments, multiple sensor signals can be fed into an artificial intelligence and the sensor signals can include notes from a physician regarding blood pressure. In some cases, the artificial intelligence can learn to predict and refine the sensing parameters that cause stimulation. In some embodiments, a neural network can be used with sensor information, physician annotations of blood pressure, and patient symptoms, such that the neural network can increase accuracy of providing stimulation based on sensor signals.

In some embodiments, the neural network can be a layered convolutional neural network (LCNN). The LCNN can see X signals in Y times (validated by a physician), and initiate stimulation. In some cases, the LCNN can monitor a plurality of patient signals and signal patterns, and receive input indicating blood pressure (e.g., high blood pressure and/or low blood pressure). The LCNN can then evaluate the patient signals and signal patterns in comparison to the input indicating blood pressure and determine whether patterns exist corresponding to a blood pressure event or no blood pressure event. Optionally, the LCNN can determine characteristics from the patient signals and signal patterns that best indicate a blood pressure event, such that physician input is not needed to initiate stimulation. Accordingly, stimulation, and therefore treatment, can be based on physician input, automated stimulation, or stimulation based on LCNN.

In some embodiments, target disease treatment is determined, in part, by the exact anatomic site in which the device is located. In addition, the stimulation parameters (e.g., sequence and/or strength) and type of stimulation, ablation, DC current injury, or blocking current delivery can be determined. Accordingly, the devices, which incorporate feedback and both distal and proximal (sensing and downstream) electrodes allows for a precise type of energy delivery for the specific disease. With a ring electrode placed around the ansa, if a patient becomes hypotensive (as determined by the vascular sensors in the venous, arterial, subcutaneous, or other location), then a stimulatory current is induced. If there is an overshoot with too much hypertension or too much neural traffic, then a blocking current can be immediately delivered. Further, simultaneous two sequence stimulation, one targeting the admixed vagal fibers and another the sympathetic fibers, could be delivered with one being inventory and the other being stimulatory.

In some embodiments, a spreading device can be used as the delivery tool. The spreading device can be deployed through a subcutaneous sheath placed using a standard modified Seldinger-type approach except not into the vascular space. Once the subcutaneous space is entered, then the spreading device has a forward facing ultrasound sensor, Doppler probes, and closely-spaced bipolar electrodes serving as its visual sensor. The tip can be opened and closed and moved forward either with manual pressure or radiofrequency or other energy delivery to obtain hemostasis and move the device forward. By using the Doppler and ultrasound, the arterial venous system is avoided, and the sensed neural signals as well as visual data from the 2D component of the ultrasound sensor used to identify the stellate ganglion and/or the ansa subclavius. When the target is reached, the spreading tool can be closed with the electrodes that were used for detection clamped on to the neural structure of interest. Optionally, the rest of the tool can then be detached by rotation or other mechanism leaving behind the required electrode and lead.

In some embodiments, the devices can use an electrode design that can be placed via the vasculature to stimulate the stellate ganglion and/or the ansa subclavius. In some cases, a simple or off-the-shelf electrode design placed via the vasculature to stimulate the stellate ganglion and/or the ansa subclavius would be insufficient. For example, arterial system electrodes may thrombose. As a result, the devices can include a stented electrode placed in the junction of the subclavian artery and its branches that can be wirelessly stimulated (e.g., from the skin surface or a similar device), but with the computer diagnostics and battery placed in the adjacent venous system. In addition, painful stimulation of surrounding structure may occur with a simple monopolar type of stimulation. Accordingly, in some cases, paired devices can be used in the surrounding venous structures and the subcutaneous space which we access via the spreader so as to minimize the field of stimulation and thus minimize extra neural stimulation. Further, the device placed in the subcutaneous tissue may serve as a current inducer to stimulate from the stent, thus creating a bipolar vector for stellate stimulation or ansa subclavius stimulation.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

What is claimed is:

1. A method of modulating hemodynamic parameters of a patient, the method comprising:
positioning a device comprising an electrode proximal to a stellate ganglion; and
delivering reversible electroporation having a frequency of 5 to 15 hertz via the electrode to the stellate ganglion.

2. The method of claim 1, wherein delivering the reversible electroporation via the electrode comprises delivering the electroporation with a pulse width, frequency, or output voltage configured to decrease a blood pressure of the patient.

3. The method of claim 2, wherein the pulse width is 1 to 3 milliseconds.

4. The method of claim 1, wherein delivering the reversible electroporation via the electrode comprises inhibiting neural activity.

5. The method of claim 1, wherein delivering the reversible electroporation via the electrode comprises temporarily inhibiting neural activity.

6. The method of claim 1, further comprising securing the device proximal to the stellate ganglion by performing at least one of:
screwing a portion of the device into the stellate ganglion;
screwing a portion of the device into tissue proximal the stellate ganglion;
securing the device proximal the stellate ganglion via a barb;
securing the device proximal the stellate ganglion via a hook; or
clamping a portion of the device around the stellate ganglion.

7. The method of claim 1, further comprising positioning a distal portion of the electrode proximal a portion of a heart of the patient, wherein the delivering the reversible electroporation via the electrode further comprises delivering the electroporation via the distal portion of the electrode.

8. The method of claim 7, further comprising sensing a change in blood pressure of the patient via the distal portion of the electrode.

9. The method of claim 1, further comprising sensing a change in blood pressure of the patient via one of a pressure sensor or plethysmograph.

10. The method of claim 9, further comprising determining the device was positioned in a correct direction based on the change in the blood pressure of the patient.

11. A method of modulating hemodynamic parameters of a patient, the method comprising:
positioning a device comprising an electrode to a first position proximal to a stellate ganglion;
sensing a change in a blood pressure of the patient; and
delivering a first reversible electroporation having a frequency of 5 to 15 hertz via the electrode to the stellate ganglion based on the change detected in the blood pressure of the patient.

12. The method of claim 11, further comprising delivering a second reversible electroporation via the electrode to the stellate ganglion based on the change detected in the blood pressure of the patient.

13. The method of claim 11, further comprising:
repositioning the device to a second position proximal to the stellate ganglion; and
delivering a second reversible electroporation via the electrode to the stellate ganglion based on the change detected in the blood pressure of the patient.
